(19) United States
(12) Patent Application Publication
(10) Pub. No.: US 2009/0105529 A1
(43) Pub. Date: Apr. 23, 2009

(54) Sex toy using internal reciprocating weights

(76) Inventor: Alan Crarer, Pacifica, CA (US)

Correspondence Address: Design Collective LLC, 2261 Market Street, #273, San Francisco, CA, 94114, US

(21) Appl. No.: 12/196,787

(22) Filed: Aug. 22, 2008

Related U.S. Application Data
(60) Provisional application No. 60/965,993, filed on Aug. 24, 2007.

(51) Int. Cl. A61F 5/00; A61H 19/00

(52) U.S. Cl. 600/38; 601/101

(57) ABSTRACT

A sex toy that uses an enclosed, internal, mechanism of reciprocating weights to induce a back and forth, or rocking, motion on the device. The back and forth motion may be used alone, or in concert with other features common to sex toys, such as; vibration, compression and suction.

The embodiment for men is roughly the shape of a donut (or torus) and sized large—to fit the majority of subjects. The embodiment for men also incorporates geometry allowing a variety of different liners to be inserted. These liners may be made from a number of different materials and provide a variety of sensations and fits.

The women's version is cylindrical with roughly spherical ends.

The user of the device is prevented from touching, interfering with, or damaging the moving parts—as they are enclosed inside the device. The speed of reciprocation can be adjusted as desired.

### BACKGROUND OF THE INVENTION

1. Field of the Invention

The present invention relates generally to devices that are used, by men and women, for sexual stimulation. More specifically, it relates to a device designed to create an approximation of back and forth motion, either alone or in concert with other sensations, without requiring the use of one's hands.

2. Background of the Invention

Sex toys that incorporate back and forth motion typically use a mechanism wherein moving components are in direct physical contact with the user. Those systems tend to encounter large variations in friction and resistance, which in turn can result in significant stress on the drive motor and associated moving parts. Subsequently, they tend to have short life-spans before becoming worn, damaged or failing to operate.

Additionally, sex toys that incorporate back and forth motion typically require the device to be held stationary, with respect to the user, while a specific portion of the device oscillates back and forth against variable friction at the user/device interface. By using the momentum of the internal mechanism to impart back and forth motion on the device as a whole, the present invention may be used without the need for anchoring or the use of one's hands.

The motivation behind the present invention is the creation of a device that approximates back and forth motion using an enclosed and protected mechanism that may be used “hands free”. By using the momentum of enclosed reciprocating weights, to approximate back and forth motion for the whole device, the load on the motor and mechanism are easily controlled and predictable, no anchoring is required, and the user cannot damage the moving parts or drive system during normal use.

### SUMMARY OF THE INVENTION

By the present invention, a sex toy using enclosed reciprocating weights to induce back and forth motion is disclosed. The sex toy provides sexual stimulation, to men and/or women, by approximating back and forth motion. Furthermore, the back and forth motion may be used alone or in combination with other effects such as; vibration, compression and suction. The reciprocating motion of the weights is derived from: an electric motor, a gear or pulley system, a rotating crank shaft and connecting rods. All moving parts are enclosed within the device.

Accordingly, one of the objects of the present invention is to provide back and forth motion, in a sex toy, that can be used without the use of one's hands and is not prone to failure or premature wear of moving parts.

In View of the above-mentioned and other objects, all of which will become more readily understood as the nature of the present invention is better understood, the invention comprises in the novel combination and arrangement of the parts hereinafter more fully described, illustrated, and claimed with reference being made to the attached drawings.

### BRIEF DESCRIPTION OF THE DRAWINGS

FIG. 1: Reciprocating weight mechanism

FIG. 2: Main assembly and outer enclosure

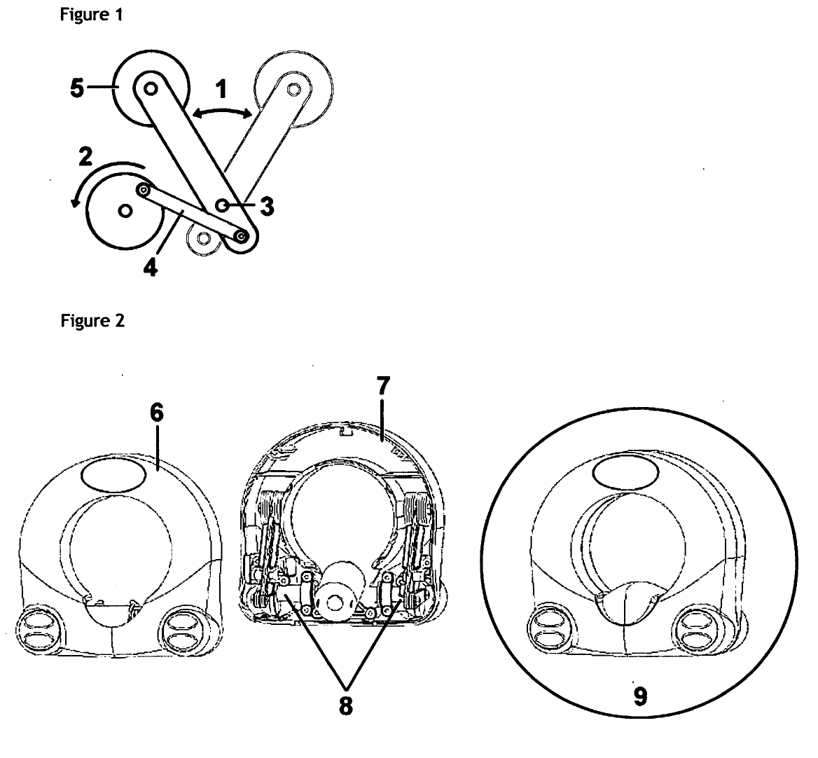

### DETAILED DESCRIPTION OF THE PREFERRED EMBODIMENT

The present invention will be seen to relate to a sex toy 9 using enclosed reciprocating weights 5 to induce back and forth motion. An electric motor transmits radial motion through a gear or pulley system into a rotating crankshaft 2. The crankshaft 2 converts that radial motion into reciprocating 1 motion using a connecting rod 4. The distance and curvature of the reciprocating 1 motion is set by the position of the pivot 3, relative to the weights 5. The assembly of moving parts 8 is enclosed between the front 6 and rear 7 enclosures.

It is to be understood that the present invention is not limited to the sole embodiment described above, but encompasses any and all embodiments within the scope of the disclosure and the following claims;

I claim:

1. A sex toy, or masturbatory aid, comprising an enclosed mechanism of reciprocating, or shifting, weights that induces a back and forth, or rocking motion on the device.

2. A sex toy, or masturbatory aid, that operates without the need to secure, hold, or anchor the device—improving simplicity and allowing the user's hands to be available for controlling visual stimulation or performing other tasks.

3. A sex toy as in claim 1, wherein said enclosed mechanism induces back and forth, or rocking, motion on the device as a whole—as opposed to existing devices wherein the device must be held stationary while a portion of the device oscillates back and forth against varying friction. Said device may operate without the need to secure, hold, or anchor said device—improving simplicity and allowing the user's hands to be available for controlling visual stimulation or performing other tasks.

4. A sex toy, or masturbatory aid, that incorporates a plurality of functions such as: back and forth (or stroking motion), vibration, compression, suction, and thermal manipulation, in order to provide a matrix of adjustments, combinations and sensations.

5. A sex toy as in claim 1, wherein said mechanism is enclosed within the device so as to discourage inadvertent damage to the device, or discomfort to the user, during regular operation.

6. A sex toy as in claim 1, that receives control signals from an external personal computer. The control signals may be derived from a plurality of applications such as: custom applications, video productions, games and internet community sites.

7. A sex toy as in claim 2, that receives control signals from an external personal computer. The control signals may be derived from a plurality of applications, including those requiring the user's hands to be available, such as: custom applications, video productions, games and internet community sites.

8. A sex toy as in claim 1, wherein the device is designed for male stimulation.

9. A sex toy as in claim 2, wherein the device is designed for male stimulation.

10. A sex toy as in claim 1, wherein the device is designed for female stimulation.

11. A sex toy as in claim 2, wherein the device is designed for female stimulation.

12. A sex toy as in claim 4, wherein the device is designed for male stimulation.

13. A sex toy as in claim 4, wherein the device is designed for female stimulation.

14. A sex toy as in claim 6, wherein the device is designed for male stimulation.

15. A sex toy as in claim 7, wherein the device is designed for male stimulation.

16. A sex toy as in claim 6, wherein the device is designed for female stimulation.

17. A sex toy as in claim 7, wherein the device is designed for female stimulation.
